(19) United States
(12) Patent Application Publication
(10) Pub. No.: US 2026/0207043 A1
(43) Pub. Date: Jul. 23, 2026

(54) APPARATUS AND METHOD FOR TREATING STRESS URINARY INCONTINENCE

(71) Applicant: ATHENA UROLOGIC, LLC, White Plains, NY (US)

(72) Inventor: Nicole FLEISCHMANN, White Plains, NY (US)

(21) Appl. No.: 19/135,967

(22) PCT Filed: Dec. 5, 2023
(86) PCT No.: PCT/US2023/082438

§ 371 Date: Jun. 5, 2025

Related U.S. Application Data
(60) Provisional application No. 63/386,115, filed on Dec. 5, 2022.

(51) Int. Cl. A61B 1/307; A61B 1/00; A61B 1/04; A61B 17/00

(52) U.S. Cl. CPC A61B 1/307; A61B 1/00082; A61B 1/04; A61B 17/00; A61B 2017/00805; A61B 2217/005; A61B 2217/007

(57) ABSTRACT

An apparatus and method for treating stress urinary incontinence is provided. The apparatus includes a conduit connected to a connection member. The connection member includes a visualization port that receives a portion of an endoscope. A fluid inflow port communicates with an irrigation source, and a fluid outflow port communicates with a suction source. A needle port receives a portion of a needle that is attachable to a pre-filled syringe containing a bulking agent to be injected into the external urethral sphincter. An inflatable balloon is located on the conduit and is inflatable by supplying an inflation fluid through a balloon port on the conduit. The inflated balloon seals an interior portion of a urethra to inhibit fluid leakage. An occlusion disc is movable along an outer surface of the conduit to seal off the urethral meatus, and thus further inhibit fluid leakage from the urethra.

### CROSS REFERENCE TO RELATED APPLICATIONS

This application claims priority to and the benefit of U.S. Provisional Ser. No. 63/386,115 filed Dec. 5, 2022, which is incorporated by reference herein in its entirety for any and all purposes.

### FIELD OF THE INVENTION

The present invention generally relates to the noninvasive treatment of stress urinary incontinence, and more particularly, to a device and method for injecting a biological bulking agent into tissue of a patient for treating stress urinary incontinence.

### BACKGROUND

Urinary incontinence often occurs when the muscles of the urinary system malfunction or are weakened. Other factors, such as trauma to the urethral area, neurological injury, hormonal imbalance, or medication-related side-effects, may also cause or contribute to incontinence. Stress urinary incontinence is a type of incontinence in which a patient experiences an involuntary loss of urine that occurs due to sudden increases in intra-abdominal pressure resulting from stressful activities such as coughing, sneezing, laughing, lifting, straining, exercise, or other physical activities that put pressure on the bladder. Stress urinary incontinence is often related to hypermobility of the bladder neck or an intrinsic urethral sphincter defect. Women are especially vulnerable to stress-related urinary incontinence as a result of injuries sustained during pregnancy. For instance, stress urinary incontinence may be due to urethral support structural damage and a relaxed pelvic floor.

A variety of treatment options are currently available to treat stress urinary incontinence, including the use of external devices, behavioral therapy (such as biofeedback, electrical stimulation, or muscle exercises), injectable materials, prosthetic devices, and/or surgery. Some conservative options for management of stress urinary incontinence can include lifestyle changes, such as weight loss, smoking cessation, and modification of intake of diuretic fluids such as coffee and alcohol.

The use of slings have been found to be effective for treating stress urinary incontinence. This involves a surgical procedure in which placement of a sling stabilizes or supports the bladder neck or urethra. There are a variety of different sling procedures. In some cases, the sling is placed under the bladder neck and secured via suspension sutures to a point of attachment through an abdominal and/or vaginal incision. However, serious complications associated with sling procedures can occur, even if infrequently. Such complications include urethral obstruction, development of new urge urinary incontinence, hemorrhage, prolonged urinary retention, infection, and damage to surrounding tissue and sling erosion.

A minimally invasive alternative to the surgical procedure is the injection of a bulking agent into the proximal urethra, that is the region of the internal urinary sphincter (IUS). Although, such injections are less invasive and involve less risk of serious complications than surgery, they are less effective than other typical treatments, such as a midurethral sling (MUS), and often do not fully alleviate the urinary incontinence. Traditional bulking agents are in the class of particulate compounds, solid microparticles in an absorbable liquid or gel carrier which can expand the submucosal tissues to narrow the opening of the bladder neck causing a reduction in urine leakage. Over time, the bulking effect is lost due to absorption of the carrier gel leaving only the inflammatory microparticles remaining. In these cases, incontinence returns and the injection needs to be repeated. Furthermore, the remaining particles in all of these formulations are immunogenic and can lead to rare complications such as abscess formation and urethral erosion requiring implant removal. Due to these risks, and the limited success and longevity of such treatments, the use of bulking agents to treat stress incontinence in the US has been sporadic and reserved for only specific cases. More recently use of polyacrylamide hydrogel (PAHG) (BULKAMID® (Axonics, Contura)), a nonparticulate bulking agent, has gained popularity for use in this approach.

An example of a conventional sheath device for injecting a bulking agent for the treatment of stress urinary incontinence is depicted in FIGS. 1A and 1B. The conventional sheath device 1 is suitable for endoscopic instruments la and comprises an elongated tubular member 2 having a proximal end 3, a distal open end 4, and at least one fluid channel extending therebetween. A needle port 5 may be provided at the proximal end 3 of the tubular member for receiving a needle 5a and a pre-filled syringe 5b containing a bulking agent. A flushing unit 6 is connected to the proximal end 3 of the tubular member 2 and comprises a proximal open end 7 suitable for receiving an endoscopic instrument, a fluid inlet 8 connected to inflow tubing 8a, and a fluid outlet 9 connected to outflow tubing 9a. The connection between the tubular member 2 and the flushing unit 6 allows for axial rotation of the tubular member in relation to the flushing unit.

There remains a need for developing novel strategies for treating stress urinary incontinence that are effective and have an acceptable risk profile and recovery period. There is also a clear and substantial need for an improved minimally invasive bulking agent treatment for stress urinary incontinence that can be performed in an outpatient setting, such as a doctor's office. There is also a need for a device for treating stress urinary incontinence that prevents or minimizes leakage of irrigation fluid or bladder drainage from the urethra during the injection of the bulking agent into the patient's sphincter. There is also a need for a device that has the ability to measure the urethral closure pressure of the external urinary sphincter to determine continence. There is also a need for a delivery system that has increased efficacy and ease of use by physicians. The present invention solves these and other problems.

### SUMMARY

The foregoing needs are met, to a great extent, by the present invention of an apparatus and method for treating stress urinary incontinence of a patient. The apparatus comprises an elongated conduit having a proximal end, a distal end defining an opening, and a main lumen extending from the proximal end to the distal end; a connection member connected to the proximal end of the elongated conduit, the connection member including a visualization port configured to receive a portion of a camera unit, a fluid inflow port configured to communicate with an irrigation fluid source, a fluid outflow port configured to communicate with an irrigation fluid reservoir and/or a fluid suction source, and a needle port configured to receive a portion of a needle; an expandable member disposed on the elongated conduit proximate the distal end of the elongated conduit, the expandable member configured to move between an unexpanded state and an expanded state, and the expandable member configured to seal an interior portion of a urethra of the patient when the expandable member is in the expanded state; and an occlusion disc slidable along an outer surface of the elongated conduit, the occlusion disc configured to inhibit fluid leakage from the urethra of the patient.

According to another aspect of the present invention, the connection member is configured to axially rotate relative to the elongated conduit.

According to another aspect of the present invention, a fluid inflow lumen may extend from the fluid inflow port to the distal end of the elongated conduit.

According to another aspect of the present invention, a fluid outflow lumen may extend from the fluid outflow port to the distal end of the elongated conduit.

According to another aspect of the present invention, the expandable member comprises an inflatable balloon.

According to another aspect of the present invention, the elongated conduit further comprises an inflation port disposed proximate the proximal end of the elongated conduit.

According to another aspect of the present invention, the elongated conduit further comprises an inflation lumen configured to provide fluid communication between the inflation port and the expandable member.

According to another aspect of the present invention, the fluid outflow port further comprises a valve configured to selectively open and close the fluid outflow port.

According to another aspect of the present invention, the valve includes a stopcock.

According to another aspect of the present invention, the elongated conduit is tubular.

According to another aspect of the present invention, a depth indicator may be disposed on the elongated conduit proximate the expandable member.

According to another aspect of the present invention, the depth indicator comprises an annular indicator line disposed around a circumference of the elongated conduit.

According to another aspect of the present invention, the distal end of the elongated conduit includes an atraumatic surface.

According to another aspect of the present invention, the occlusion disc comprises an elastomeric O-ring.

According to another aspect of the present invention, a method for treating stress urinary incontinence in a patient comprises: inserting a portion of an elongated conduit of a sheath into a urethra of a patient; irrigating a bladder of the patient with irrigation fluid from a fluid inflow port of the sheath; draining the bladder through a fluid outflow port of the sheath; adjusting a position of the portion of the elongated conduit within the urethra until a depth indicator on the elongated conduit aligns with an opening of the urethral meatus of the patient; inflating an expandable member in the fossa navicularis of the patient; moving an occlusion disc along an outer surface of the elongated conduit until occlusion disc abuts a skin of the urethral meatus; and injecting a bulking agent through a needle into an external urethral sphincter of the patient.

According to another aspect of the present invention, the bulking agent comprises a polyacrylamide hydrogel.

According to another aspect of the present invention, the expandable member comprises an inflatable balloon.

According to another aspect of the present invention, the depth indicator comprises an annular indicator line disposed around a circumference of the elongated conduit.

According to another aspect of the present invention, the sheath further comprises a connection member connected to the elongated conduit.

According to another aspect of the present invention, the method further comprises attaching a camera unit to a visualization port of the connection member.

According to another aspect of the present invention, the camera unit comprises a cystoscope.

According to another aspect of the present invention, in a delivery system for administering a bulking agent into an external urethral sphincter of a patient, the delivery system comprising: an elongated conduit, a connection member connected to a proximal end of the elongated conduit, the connection member including a visualization port configured to receive a portion of a camera unit, a fluid inflow port configured to communicate with an irrigation fluid source, a fluid outflow port configured to communicate with an irrigation fluid reservoir and/or a fluid suction source, and a needle port configured to receive a portion of a needle, the improvement comprising: an expandable member disposed on the elongated conduit, the expandable member configured to move between an unexpanded state and an expanded state, and the expandable member configured to seal an interior portion of a urethra of the patient when the expandable member is in the expanded state; and an occlusion disc slidable along an outer surface of the elongated conduit, the occlusion disc configured to inhibit fluid leakage from the urethra of the patient.

According to another aspect of the present invention, in a method for administering a bulking agent into to an external urethral sphincter of a patient, the method comprising: attaching a camera unit to a visualization port of a connection member of a sheath comprising an elongated conduit and a connection member rotatably connected to the elongated conduit; inserting a portion of the elongated conduit into the patient's urethra; irrigating the patient's bladder with irrigation fluid from a fluid inflow port of the sheath; draining the bladder through a fluid outflow port of the sheath; adjusting a position of the portion of the elongated conduit within the urethra until a depth indicator on the elongated conduit aligns with an opening of the urethral meatus of the patient; and injecting a bulking agent through a needle into a urethral sphincter of the patient, the improvement comprising prior to irrigating the patient's bladder: inflating an expandable member in the fossa navicularis of the patient; and moving an occlusion disc along an outer surface of the elongated conduit until the occlusion disc abuts a skin of the urethral meatus.

There has thus been outlined certain embodiments of the present invention in order that the detailed description thereof herein may be better understood, and in order that the present contribution to the art may be better appreciated. There are additional embodiments of the present invention that will be described below and which form the subject matter of the claims appended hereto.

In this respect, before explaining at least one aspect of the apparatus and method for treating stress urinary incontinence in detail, it is to be understood that the apparatus and method are not limited in their application to the details of construction and to the arrangements of the components set forth in the following description or illustrated in the drawings. The apparatus and method are capable of aspects in addition to those described, and of being practiced and carried out in various ways.

Also, it is to be understood that the phraseology and terminology employed herein, as well as the abstract, are for the purpose of description and should not be regarded as limiting.

As such, those skilled in the art will appreciate that the conception upon which this invention is based may readily be utilized as a basis for the designing of other structures, methods, and systems for carrying out the several purposes of the present invention. It is important, therefore, that the claims be regarded as including such equivalent constructions insofar as they do not depart from the spirit and scope of the invention.

### BRIEF DESCRIPTION OF THE DRAWINGS

In order that the present invention may be readily understood, aspects of the delivery system are illustrated by way of examples in the accompanying drawings, in which like parts are referred to with like reference numerals throughout. The file of this patent or application contains at least one drawing/photograph executed in color. Copies of this patent or patent application publication with color drawing(s)/photograph(s) will be provided by the Office upon request and payment of the necessary fee.

FIG. 1A illustrates a conventional delivery system in an unassembled state.

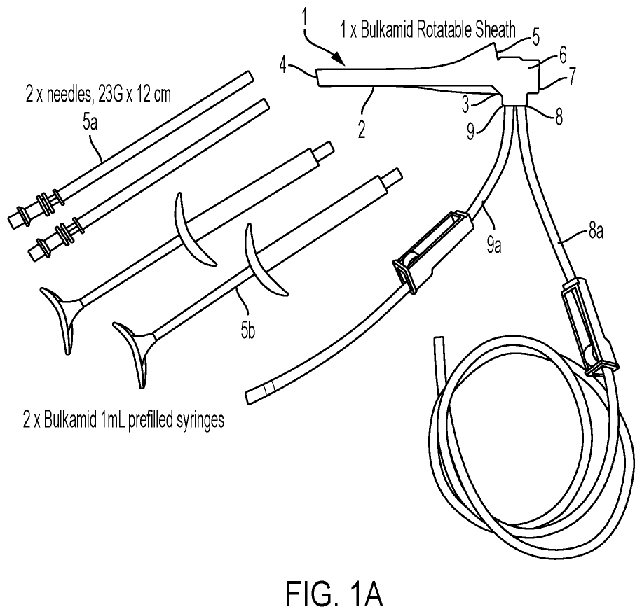

FIG. 1B illustrates the conventional delivery system in an assembled state.

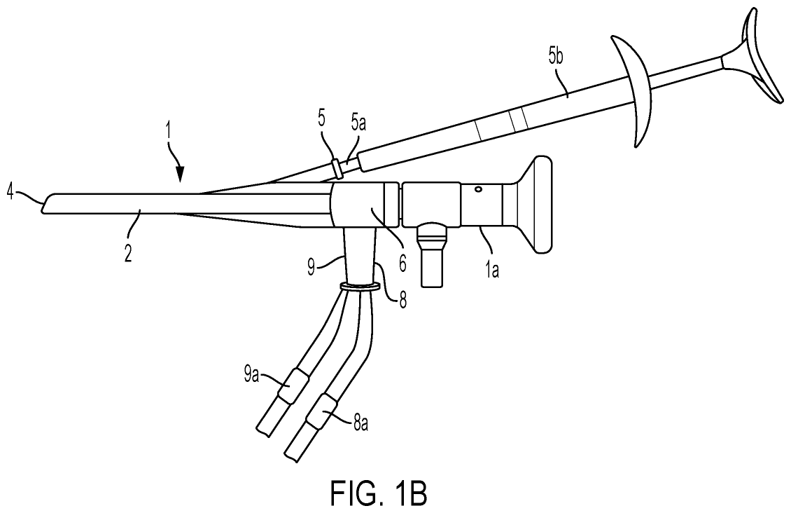

FIG. 2 illustrates a side view of a sheath of a delivery system according to the present disclosure.

FIG. 3 illustrates a side view of another implementation of the sheath according to the present disclosure.

FIG. 4 illustrates a side view of the delivery system including a sheath connected to a needle and a cystoscope according to the present disclosure.

FIG. 5 illustrates a side view of another embodiment of the sheath 10′ according to the present disclosure having upper and lower injection ports and a proximal end that does not rotate.

FIGS. 6A and 6B illustrate a side view of a sheath 10′according to the present disclosure having upper and lower injection ports with a camera unit assembled with the sheath (FIG. 6A) and the camera unit separate from the sheath (FIG. 6B).

FIGS. 7A and 7B illustrate a top view of another embodiment of the sheath 10′ according to the present disclosure having upper and lower injection ports shown with an obturator in place (FIG. 7A), and alone with the obturator removed (FIG. 7B). The dotted lines indicate the direction of placement of the obturator within the main lumen of the sheath. The obturator can aid in placement of the sheath.

FIGS. 8A and 8B illustrate a side view of an embodiment of the sheath 10′of the present disclosure having upper and lower injection ports with the obturator in place (FIG. 8A) and the obturator alone (FIG. 8B).

FIGS. 9A and 9B illustrates a right-side view (FIG. 9A) and a left side view (FIG. 9B) of an embodiment of sheath 10′ the present disclosure having upper and lower injection ports.

FIG. 10 illustrates an anatomy of a subject during treatment of stress urinary incontinence in accordance with the present disclosure.

FIG. 11 illustrates an exemplary cross-sectional view of the main lumen of a sheath of the present disclosure in place showing the lens of the camera unit in the center of the main lumen, and upper and lower injection tracts that connect to the upper and lower injection ports, respectively. Also illustrated are inflow and outflow ports in the sheath for flow of fluid injected during placement of the sheath in a subject. The dotted line through the small image of the sheath shows the cut line for the cross-sectional view.

### DETAILED DESCRIPTION

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

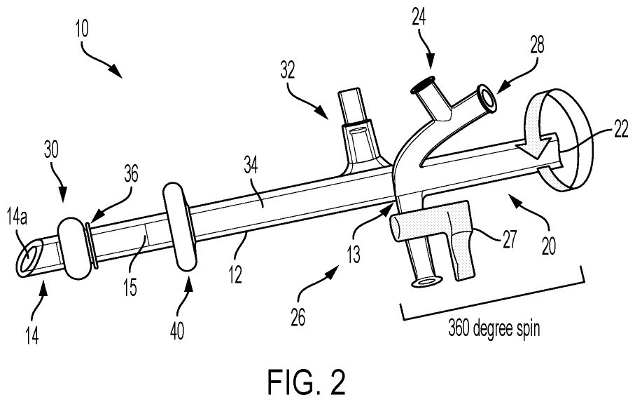

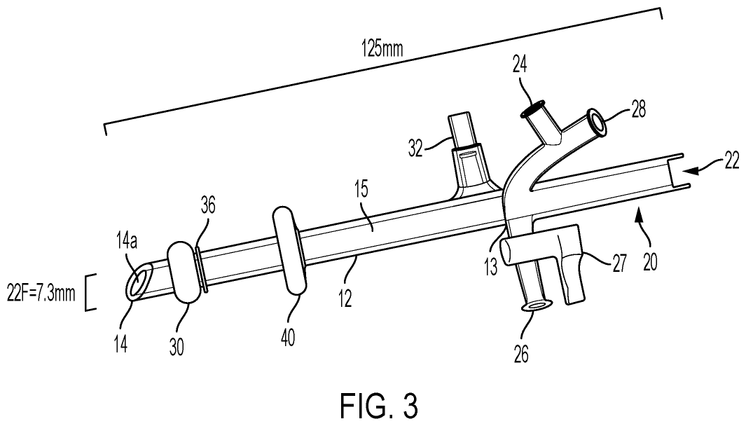

The present invention is directed to an apparatus and method for treating stress urinary incontinence in a patient. Referring to FIGS. 2 and 3, a sheath 10 comprises an elongated conduit 12 having a proximal end 13, a distal end 14, and a main lumen 15 extending from the proximal end to the distal end. The distal end defines an opening 14a for the passage of an irrigation fluid and one or more medical instruments, such as a needle 50 and a camera unit 60. According to some aspects, the distal end 14 of the elongated conduit 12 may also include an atraumatic surface. Also according to some aspects, the elongated conduit 12 is tubular. The sheath 10 further comprises a connection member 20 connected to the proximal end 13 of the elongated conduit 12. The sheath can include any suitable length as understood in the art including for example, up to 50 mm, from about 50 mm to about 100 mm, from about 100 mm to about 120 mm, from about 120 mm to about 130 mm, from about 130 mm to about 140 mm, from about 140 mm to about 150 mm, from about 150 mm to about 160 mm, from about 160 mm to about 170 mm, from about 170 mm to about 180 mm, from about 180 mm to about 190 mm, from about 190 mm to about 200 mm, from about 200 mm to about 210 mm, from about 210 mm to about 220 mm, from about 220 mm to about 230 mm, from about 230 mm to about 240 mm, from about 240 mm to about 250 mm, from about 250 mm to about 260 mm, from about 260 mm to about 270 mm, from about 270 mm to about 280 mm, from about 280 mm to about 290 mm, from about 290 mm to about 300 mm, including any and all increments therebetween. In some implementations, a length of the sheath may be approximately 125 mm, as depicted in FIG. 3. This length is well-suited for use of the sheath on the female anatomy. In other implementations, the length of the sheath 10, and more particularly, the length of the elongated conduit 12 may be longer than 125 mm for use on the male anatomy, as the distance between the urethral meatus and the external urethral sphincter is longer for males than it is for females. For instance, the length of the sheath may be approximately 22 cm to use over 30 cm 0 degree lens.

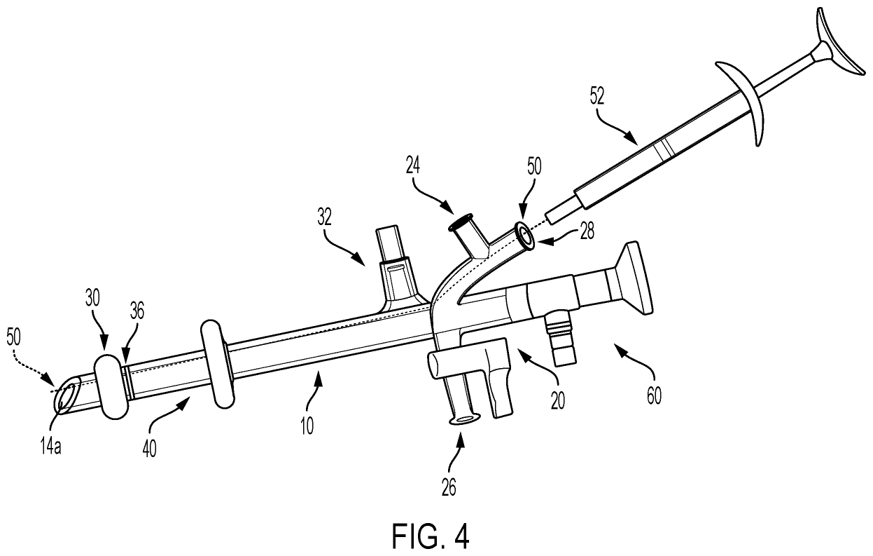

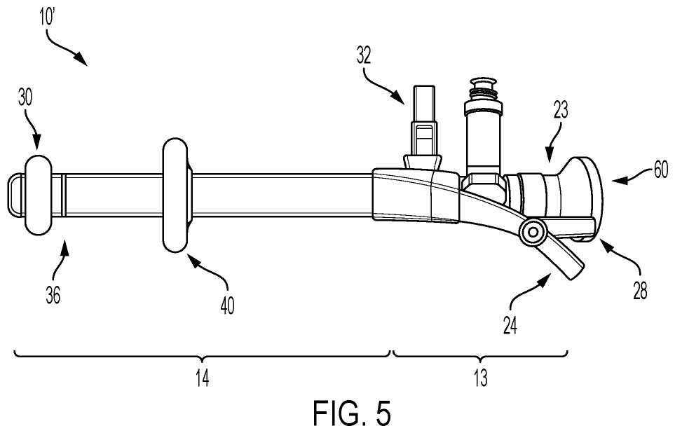

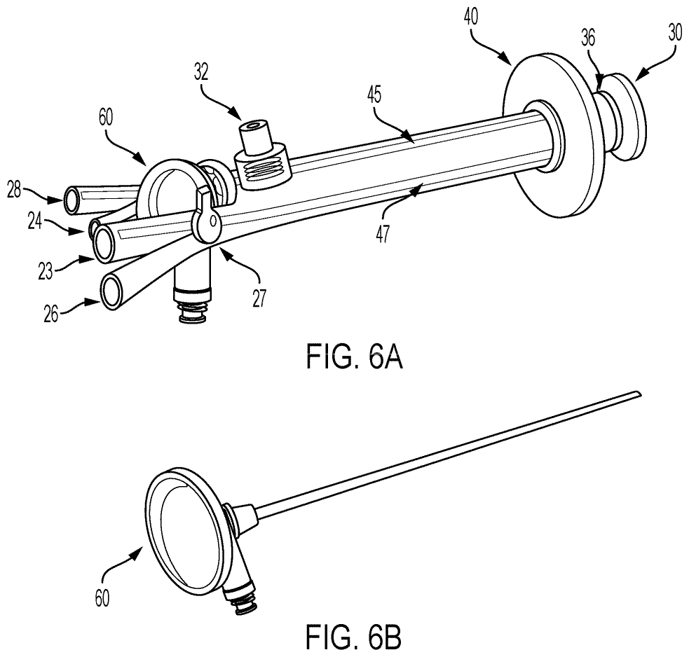

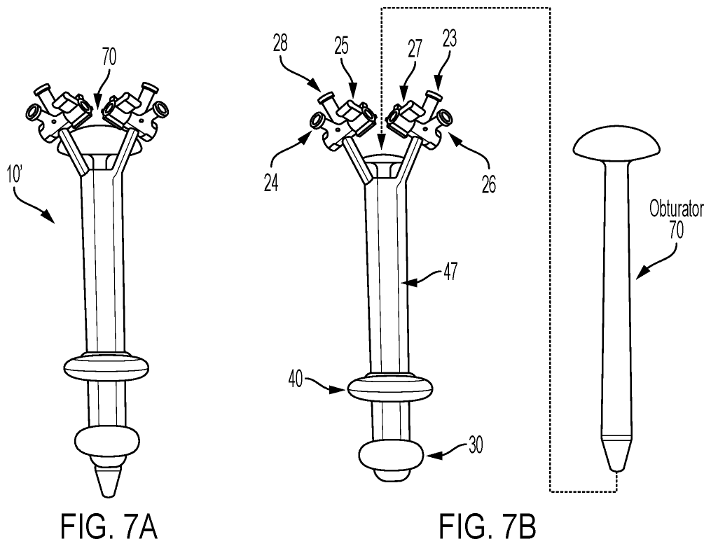

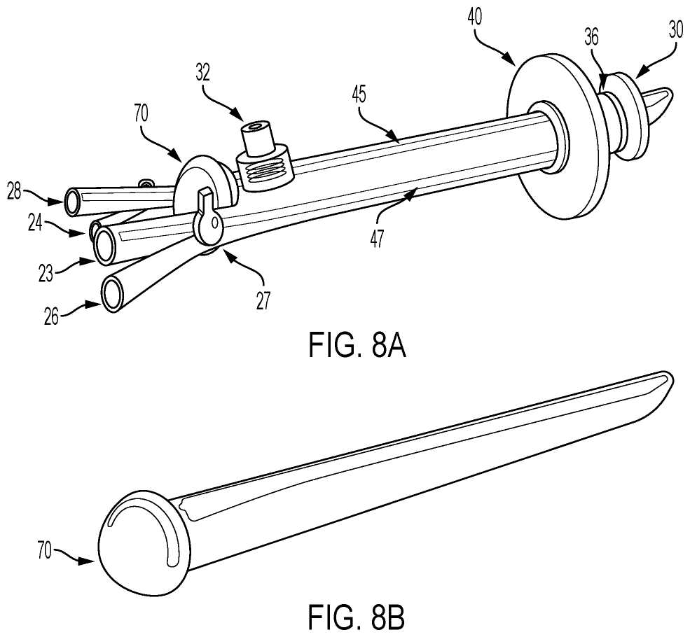

As shown in FIG. 4, the connection member 20 includes a visualization port 22 configured to receive a portion of a camera unit 60, such as a cystoscope or other type of endoscope. According to some aspects, the visualization port 22 is further configured to releasably engage the camera unit 60. The visualization port 22 communicates with the main lumen 15 of the elongated conduit 12 to provide a pathway for a portion of the camera unit 60 to travel through to the opening 14a at the distal end 14. In some implementations, the connection member 20 is configured to axially rotate 360° relative to the elongated conduit 12, wherein a longitudinal axis of the elongated conduit 12 remains in alignment with a longitudinal axis of the visualization port 22 during such rotation. In some implementations, the connection member 20 is fixed and does not rotate. In some embodiments, such as those shown in FIGS. 5-9, sheath 10′ includes a proximal end 13 that connects continuously with distal end 14.

The connection member 20 further includes a fluid inflow port 24 configured to communicate with an irrigation fluid source for supplying clean irrigation fluid to the patient during a procedure. Such irrigation fluid may include saline or other biocompatible cleaning liquid. The connection member 20 may also include a fluid outflow port 26 configured to communicate with an irrigation fluid reservoir for collecting contaminated irrigation fluid from the patient during the procedure. In some implementations, the fluid outflow port 26 may be configured to communicate with a suction source for aspirating the contaminated irrigation fluid from the patient. The fluid outflow port may include a valve 27 configured to selectively open and close the fluid outflow port. According to some aspects, the valve 27 may include a stopcock.

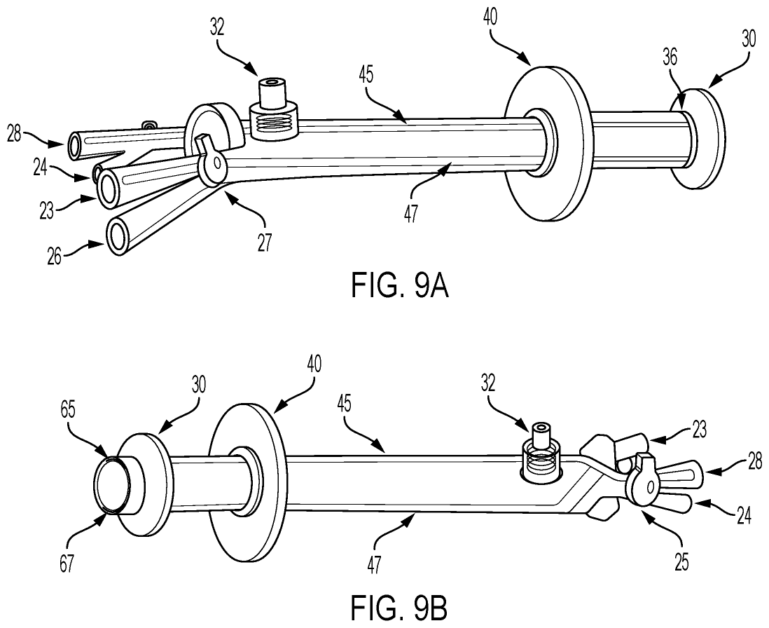

The connection member 20 also includes a needle port 28 configured to receive a portion of a needle 50 for attachment to a pre-filled syringe 52 containing a bulking agent. According to some aspects, the needle port 28 may include a septum, such as a self-sealing membrane, configured to be penetrated by a sharp tip of the needle to allow passage of the needle therethrough while preventing fluid leakage from the needle port. The bulking agent may include any suitable bulking agent or similar composition as understood in the art. For example, in some implementations the bulking agent can include a hydrogel. The hydrogel can include a polyacrylamide hydrogel (PAHG), such as BULKAMID®. In some implementations, the elongated conduit 12 may further include a fluid inflow lumen in fluid communication with the fluid inflow port 24 of the connection member 20. The fluid inflow lumen 45 can extend the length of elongated conduit 12. As shown in FIG. 9, the fluid inflow lumen can include one or more distal openings 65 for delivering a fluid to the distal end of the sheath. In other implementations, the elongated conduit 12 may further include a fluid outflow lumen in fluid communication with the fluid outflow port of the connection member 20.

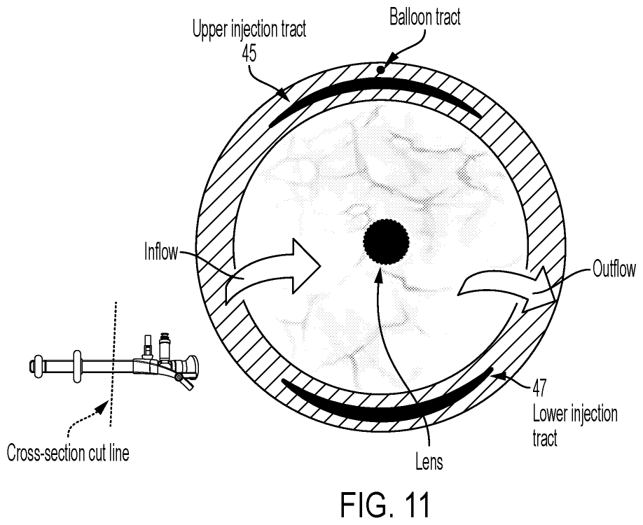

As shown in FIGS. 5-9, in some implementations, sheath 10′ includes a proximal end 13 and a distal end 14, where proximal end includes an inflow hub having a fluid inflow port 24, a first needle port 28, and inflow valve 25. Inflow valve 25 can include a stopcock. Fluid inflow port 24 can be fluidly connected to an inflow lumen or tract 45 extending the length of elongated conduit 12 an forming an inflow opening 65 (shown in FIG. 9B) at distal end 14 of sheath 10′. Inflow valve 25 can be configured to selectively open and close the fluid inflow port 24. For example, the fluid inflow port 24 can be configured to a closed position when needle port 28 receives a portion of a needle 50 attached to a pre-filled syringe 52 containing a bulking agent. Alternatively, in some embodiments, inflow valve 25 can be configured to an opened position so that fluid inflow port 24 can receive one or more fluids while a bulking agent is injected into needle port 28. The fluid inflow port 24 can be configured to communicate with an irrigation fluid source for supplying clean irrigation fluid to the patient during a procedure. Such irrigation fluid may include saline or other biocompatible cleaning liquid. The irrigation fluid source can include a high-pressure fluid flow system for introducing high pressure fluid flow through inflow lumen 45 to inflow opening 65. In some embodiments, the needle port 28 includes a septum, such as a self-sealing membrane, configured to be penetrated by a sharp tip of the needle 50 to allow passage of the needle therethrough while preventing fluid leakage from the needle port. A distal end of inflow lumen 45 can include one or more distal inflow openings 65. In some embodiments, the distal end of inflow lumen 45 can also and/or instead include one or more openings for introducing one or more fluids into the distal end of main lumen 15, shown in FIG. 11.

Proximal end 13 of the sheath 10′ can also include an outflow hub having a fluid outflow port 26, a second needle port 23, and outflow valve 27. Outflow valve can include a stopcock. Fluid outflow port 26 can be fluidly connected to an outflow lumen 47 extending the length of elongated conduit 12 an forming an opening 67 (shown in FIGS. 9A and 9B) at distal end 14 of sheath 10′. Outflow valve 27 can be configured to selectively open and close the fluid outflow port 26. For example, the fluid outflow port 26 can be configured to a closed position when needle port 23 is receiving a portion of a needle 50 attached to a pre-filled syringe 52 containing a bulking agent. Alternatively, in some embodiments, inflow valve 27 can be configured to an opened position so that fluid outflow port 26 can receive or drain one or more fluids while a bulking agent in injected into second needle port 23. The one or more received or drained fluids can include one or more contaminated fluids. In some implementations, the fluid outflow port 26 may be configured to communicate with a suction source for aspirating the contaminated irrigation fluid from the patient. In some embodiments, the second needle port 23 includes a septum, such as a self-sealing membrane, configured to be penetrated by a sharp tip of the needle 50 to allow passage of the needle therethrough while preventing fluid leakage from the needle port. Distal end of outflow lumen 47 can include one or more distal openings 67. In some embodiments, distal end of inflow lumen 47 can also and/or instead include one or more openings, shown in FIG. 11 as inflow and outflow openings, for collecting or receiving one or more fluids into the distal end of main lumen 15.

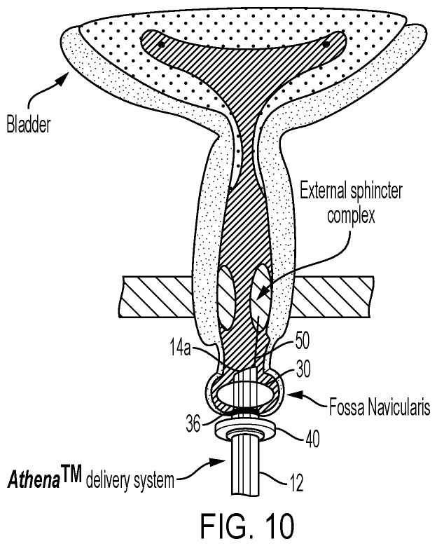

Embodiments of the sheath (10 and 10′) further include an expandable member 30 disposed on the elongated conduit 12 proximate the distal end 14 of the elongated conduit. According to some aspects, the expandable member includes an inflatable balloon. The expandable member is configured to alternate between an unexpanded state (i.e., in which the balloon is uninflated) and an expanded state (i.e., in which the balloon is inflated). The expandable member 30 is operable to seal an interior portion of a urethra of patient when the expandable member is in the expanded state. In some implementations, the expandable member 30 is expanded once positioned within or in close proximity to the fossa navicularis, as shown in FIG. 10.

The elongated conduit 12 further comprises an inflation port 32 disposed proximate the proximal end 13 of the elongated conduit. The inflation port 32 is configured to communicate with an inflation fluid source. The elongated conduit 12 comprises an inflation lumen 34 configured to provide communication for the inflation fluid between the inflation port 32 and the expandable member 30. Such inflation fluid may comprise a liquid and/or a gas. For instance, the liquid may include water, saline, or the like. The gas may include air, a blood-gas mixture, oxygen, nitrogen, carbon dioxide, or other suitable gases or gas mixtures. A liquid-gas mixture may predominantly include liquid mixed with gas bubbles.

A depth indicator 36 may also be disposed on the elongated conduit proximate the inflatable balloon. According to some aspects, the depth indictor 36 may include an indicator line extending annularly around a circumference of the tubular elongated conduit 12. The indicator line may be colored red, or some other color suited to stand out to a practitioner for ease of distinguishing a depth of insertion of the elongated conduit into the patient's urethra.

A movable occlusion disc 40 is also provided on the elongated conduit 12. In particular, the occlusion disc 40 configured to slide along an outer surface of the elongated conduit 12 for sealing an opening of the urethra when a portion of the elongated conduit is inserted in the urethra, thus inhibiting fluid leakage from the urethra of the patient during a procedure. According to some aspects, the occlusion disc may comprise an elastomeric O-ring.

During treatment of the external urethral sphincter, as will be discussed further below, there needs to be a high-pressure flow to visualize the relevant anatomy, and due to the distal position of the external sphincter, there is significant extravasation of saline fluid from the commercially available device during the procedure. In some embodiments, a high-pressure flow system can be connected to the sheath (10 and 10′). The high-pressure flow system can be attached the sheath by connecting to, for example fluid inflow port 24 and fluid outflow port 26. The high-pressure flow system can include one or more flow sensors and/or pressure sensors for measuring, detecting and/or achieving a suitable pressure for proper placement of the sheath and/or bulking agent. In some embodiments, the high-pressure flow system achieves a pressure of 60 cmH₂O. The pressure can include up to about 30 cmH₂O, from about 30 cmH₂O to about 40 cmH₂O, from about 40 cmH₂O to about 50 cmH₂O, from about 50 cmH₂O to about 60 cmH₂O, from about 60 cmH₂O to about 70 cmH₂O, from about 70 cmH₂O to about 80 cmH₂O, from about 80 cmH₂O to about 90 cmH₂O, from about 90 cmH₂O to about 100 cmH₂O, from about 100 cmH₂O to about 110 cmH₂O, from about 110 cmH₂O to about 120 cmH₂O, from about 120 cmH₂O to about 130 cmH₂O, from about 130 cmH₂O to about 140 cmH₂O, from about 140 cmH₂O to about 150 cmH₂O, including any and all increments therebetween. This leakage occurs outside of the sheath, which creates unsanitary conditions typically unsuitable for performing the procedure in an outpatient environment such as a doctor's office. As a result, the treatment would normally be relegated to being performed in a hospital environment, such an operating room equipped to disinfect and sanitize the operating space after each procedure. However, the use of the expandable member 30 and the occlusion disc 40 of the present invention helps prevent or minimize fluid leakage, thus allowing the procedure to be performed in an outpatient setting such as a doctor's office. Moreover, the sheath functions as a contained delivery system using the expandable member because the cystoscopic realignment/reconstruction of external sphincter technique (CREST) requires a high-pressure flow which causes more leakage than the traditional technique. Furthermore, maximum urethral closing pressure is measured which tells the practitioner how much pressure is required to pass through the external urethral sphincter. If the water escapes, the practitioner does not obtain a true pressure. The expandable member is operable to push the external urethral sphincter into the practitioner's view, which will aide in the teaching of the procedure to people unfamiliar with the anatomy.

### Injection Procedure

Treatment of stress urinary incontinence of a patient may be performed by providing a delivery system for administering a bulking agent, such as a PAHG, directly into an external urethral sphincter of the patient. Such a delivery system comprises the sheath 10 described above, the needle 50, the pre-filled syringe 52 containing the PAHG, and the endoscope 50. An example technique for endoscopic reconstruction of the external urethral sphincter by administering PAHG from the delivery system of the present invention comprises the following steps:
Step 1: Attach a light cable to the cystoscope 2.7×113 mm, 0° (Axonics).
Step 2: Place the cystoscope 2.7×113 mm, 0° (with camera) into the delivery system. Attach the light cable to the camera. A click will be heard when the optic is fully inserted into the sheath. Thus, the practitioner attaches the cystoscope to the visualization port 22 of the connection member 20 of the sheath 10 such that a portion of the cystoscope is disposed in the main lumen 15 of the elongated conduit. This allows the practitioner to visualize, via the cystoscope, an insertion of a portion of the elongated conduit 12 into the patient's urethra.
STEP 3: Attach the irrigation system to the delivery system via the inflow port and flush the system under normal pressure. More particularly, the fluid inflow port 24 is connected to an irrigation fluid source, and the fluid outflow port 26 is connected to an irrigation fluid reservoir and/or an irrigation fluid suction source.
STEP 4: Perform routine cystourethroscopy with the outflow port in closed position. Next open the outflow port and drain the bladder.
STEP 5: Pull the delivery system back until the indicator line reaches the skin of the urethral meatus. Under direct vision, add 2 ml of air into the balloon port and inflate the balloon in the fossa navicularis. At this point, the external sphincter complex will come into view. FIG. 10 illustrates the step of inserting a portion of the elongated conduit 12 of the sheath a distance into a female urethra until the depth indicator 36 reaches the urethral meatus. The inflation port 32 is connected to inflation source that pumps inflation fluid to the expandable member 30, such as an inflatable balloon. Fossa navicularis occurs at the location of the inflatable balloon upon inflation, thus sealing the interior channel of the urethra.
STEP 6: Insert IV bag into pressure flow bag and pump the bag to the 120 cm/H20 mark (if the bag has no mark, pump to firm). Alternatively, if no pressure bag is available, hang the IV bag at 120 cm above the patient's symphisis pubis and run wide open.
STEP 7: Remove any air bubbles from the irrigation system by opening the inflow tap. Once all air bubbles have been removed, sinch the blue occlusion disc flush with the meatus. Thus, the occlusion disc 40 is slid along the outer surface of the elongated conduit 12 in the direction toward the urethral meatus to close the opening the of the urethral meatus, and thus inhibit leakage of fluid therefrom.
STEP 8: Perform cystoscopic realignment/reconstruction of external sphincter technique (CREST) procedure according to medical guidelines.
STEP 9: When the external urethral sphincter is determined to be sealed (usually 3 ml of BULKAMID® needed), deflate the balloon port and pull the device out of the meatus. In some implementations, the bulking agent is injected directly into the external urethral sphincter of the subject at a position that is about 2-3 cm from the bladder neck. In some embodiments, the bulking agent is injected into to the circumference of the external urethral sphincter. In some embodiments, the method may comprise multiple injections of the bulking agent into the external urethral sphincter.
STEP 10: Leave 200 ml in the bladder by catheterizing the patient with 12 fr catheter.

Although FIG. 10 depicts a method of treating stress urinary incontinence in a female patient, comprising the injection of a bulking agent directly into the external urethral sphincter of the female subject, it should be appreciated that in other implementations, the method of treating stress urinary incontinence may similarly be applied to a male patient. As previously discussed above, for male subjects, a length the elongate conduit of the sheath is greater than the length of the elongate conduit of the sheath used for female subjects since the distance between the urethral meatus and the external urethral sphincter is longer for males than it is for females.

While a sample bulking agent delivery system and method of treating stress urinary incontinence has been described in terms of what may be considered to be specific aspects, the present invention is not limited to the disclosed aspects. Additional modifications and improvements to the invention may be apparent to those skilled in the art. Moreover, the many features and advantages of the disclosure are apparent from the detailed specification, and thus, it is intended by the appended claims to cover all such features and advantages of the present invention which fall within the spirit and scope of the disclosure.

Further, it is not desired to limit the disclosure to the exact construction and operation illustrated and described, and accordingly, all suitable modifications and equivalents may be resorted to, falling within the scope of the disclosure. The present disclosure should therefore be considered as illustrative and not restrictive. As such, this disclosure is intended to cover various modifications and similar arrangements included within the spirit and scope of the claims, which should be accorded their broadest interpretation so as to encompass all such modifications and similar structures.

1. An apparatus for treating stress urinary incontinence in a patient, the apparatus comprising:
an elongated conduit having a proximal end, a distal end defining an opening, and a main lumen extending from the proximal end to the distal end;
a connection member connected to the proximal end of the elongated conduit, the connection member including a visualization port configured to receive a portion of a camera unit, a fluid inflow port configured to communicate with an irrigation fluid source, a fluid outflow port configured to communicate with an irrigation fluid reservoir and/or a fluid suction source, and a needle port configured to receive a portion of a needle;
an expandable member disposed on the elongated conduit proximate the distal end of the elongated conduit, the expandable member configured to move between an unexpanded state and an expanded state, and the expandable member configured to seal an interior portion of a urethra of the patient when the expandable member is in the expanded state; and
an occlusion disc slidable along an outer surface of the elongated conduit, the occlusion disc configured to inhibit fluid leakage from the urethra of the patient.

2. The apparatus according to claim 1, wherein the connection member is optionally configured to axially rotate relative to the elongated conduit.

3. The apparatus according to claim 1, further comprising a fluid inflow lumen extending from the fluid inflow port to the distal end of the elongated conduit.

4. The apparatus according to claim 1, further comprising a fluid outflow lumen extending from the fluid outflow port to the distal end of the elongated conduit.

5. The apparatus according to claim 1, wherein the expandable member comprises an inflatable balloon.

6. The apparatus according to claim 1, wherein the elongated conduit further comprises an inflation port disposed proximate the proximal end of the elongated conduit.

7. The apparatus according to claim 6, wherein the elongated conduit further comprises an inflation lumen configured to provide fluid communication between the inflation port and the expandable member.

8. The apparatus according to claim 1, wherein the fluid outflow port further comprises a valve configured to selectively open and close the fluid outflow port.

9. (canceled)

10. The apparatus according to claim 1, wherein the elongated conduit is tubular.

11. The apparatus according to claim 1, further comprising a depth indicator disposed on the elongated conduit proximate the expandable member.

12. The apparatus according to claim 11, wherein the depth indicator comprises an annular indicator line disposed around a circumference of the elongated conduit.

13. The apparatus according to claim 1, wherein the distal end of the elongated conduit includes an atraumatic surface.

14. The apparatus according to claim 1, wherein the occlusion disc comprises an elastomeric O-ring.

15. A method for treating stress urinary incontinence in a patient, the method comprising:
inserting a portion of an elongated conduit of a sheath into a urethra of a patient;
irrigating a bladder of the patient with irrigation fluid from a fluid inflow port of the sheath;
draining the bladder through a fluid outflow port of the sheath;
adjusting a position of the portion of the elongated conduit within the urethra until a depth indicator on the elongated conduit aligns with an opening of the urethral meatus of the patient;
inflating an expandable member in the fossa navicularis of the patient;
moving an occlusion disc along an outer surface of the elongated conduit until occlusion disc abuts a skin of the urethral meatus; and
injecting a bulking agent through a needle into an external urethral sphincter of the patient.

16. The method according to claim 15, wherein the bulking agent comprises a polyacrylamide hydrogel.

17. The method according to claim 15, wherein the expandable member comprises an inflatable balloon.

18. The method according to claim 15, wherein the depth indicator comprises an annular indicator line disposed around a circumference of the elongated conduit.

19. The method according to claim 15, wherein the sheath further comprises a connection member connected to the elongated conduit.

20. The method according to claim 19, further comprising attaching a camera unit to a visualization port of the connection member, wherein the camera unit comprises a cystoscope.

21. (canceled)

22. A delivery system for administering a bulking agent into an external urethral sphincter of a patient, the delivery system comprising: an elongated conduit, a connection member connected to a proximal end of the elongated conduit, the connection member including a visualization port configured to receive a portion of a camera unit, a fluid inflow port configured to communicate with an irrigation fluid source, a fluid outflow port configured to communicate with an irrigation fluid reservoir and/or a fluid suction source, and a needle port configured to receive a portion of a needle, the improvement comprising:
an expandable member disposed on the elongated conduit, the expandable member configured to move between an unexpanded state and an expanded state, and the expandable member configured to seal an interior portion of a urethra of the patient when the expandable member is in the expanded state; and
an occlusion disc slidable along an outer surface of the elongated conduit, the occlusion disc configured to inhibit fluid leakage from the urethra of the patient.

23. (canceled)
